(19) United States
(12) Patent Application Publication
(10) Pub. No.: US 2024/0188897 A1
(43) Pub. Date: Jun. 13, 2024

(54) MACHINE LEARNING BASED HISTOPATHOLOGICAL RECURRENCE PREDICTION MODELS FOR HPV+ HEAD / NECK SQUAMOUS CELL CARCINOMA

(71) Applicants: THE UNIVERSITY OF CHICAGO, Chicago, IL (US); THE TRUSTEES OF THE UNIVERSITY OF PENNSYLVANIA, Philadelphia, PA (US)

(72) Inventors: Alexander T. PEARSON, Chicago, IL (US); James DOLEZAL, Chicago, IL (US); Devraj BASU, Philadelphia, PA (US); Robert BRODY, Philadelphia, PA (US); Jalal JALALY, Philadelphia, PA (US)

(21) Appl. No.: 18/556,328

(22) PCT Filed: Apr. 21, 2022
(86) PCT No.: PCT/US2022/025699

§ 371 Date: Oct. 19, 2023

Related U.S. Application Data
(60) Provisional application No. 63/179,091, filed on Apr. 23, 2021.

(51) Int. Cl. A61B 5/00; G06V 10/82

(52) U.S. Cl. CPC A61B 5/7267; G06V 10/82; G06V 2201/032

(57) ABSTRACT

An example embodiment involves generating tumor image tiles from images of human papillomavirus positive (HPV +) head and neck squamous cell carcinoma (HNSCC) tumors, wherein the tumor image files are respectively labelled with indicators of tumor recurrence. The example embodiment may further involve training a neural network with the tumor image files as labelled. wherein the training results in the neural network learning combinations of histology features characteristic of tumor recurrence. Further steps may involve providing further tumor image tiles to the trained neural network. the neural network generating classifications of the further tumor image tiles based on likelihood of tumor recurrence. and storing the classifications with as respectively associated with the further tumor image tiles.

### CROSS-REFERENCE TO RELATED APPLICATION

This application claims priority to U.S. provisional patent application no. 63/179,091, filed Apr. 23, 2021, which is hereby incorporated by reference in its entirety.

### BACKGROUND

Human papillomavirus positive (HPV+) head and neck squamous cell carcinomas (HNSCCs) continue to rise in incidence after being recognized as a distinct subtype of HNSCC over a decade ago. Most HPV+ HNSCCs originate from the oropharynx, which has surpassed the cervix as the leading anatomic site for HPV-related cancer in the US. In comparison to HPV-negative HNSCCs, HPV+ cases tend to arise in younger patients who lack smoking history and have more favorable oncologic outcomes. Because HPV+ HNSCCs are often cured, a therapeutic objective has been to reduce morbidity caused by their treatment with high dose radiation plus cisplatin, which often leaves life-long disabilities. Unfortunately, attempts to decrease radiation dose or supplant cisplatin use with anti-EGFR therapy have reduced survival in some trials because of a lack of adequate biomarkers to identify recurrence-prone patients. Prospective identification of such patients would exclude them from therapy de-escalation and select them for testing of novel therapies. Likewise, accurately identifying patients at lowest recurrence risk would reduce treatment-related morbidity by allowing aggressive therapy de-escalation in suitable patients.

### SUMMARY

The embodiments herein introduce machine learning based histopathological recurrence prediction models for HPV+ HNSCCs. A neural network pipeline was developed to incorporate digital pathology data and make predictions for HPV+HNSCCs recurrence following surgical resection. Such a neural network can be a form of artificial neural network (ANN), such as a deep convolutional neural network (DCNN) or some other form. Herein, the terms deep convolutional neural network (DCNN) and convolutional neural network (CNN) are used synonymously, and may refer to neural networks of various depths and arrangements.

A training data set was created based on clinical annotations and matching (anonymized) digital diagnostic pathology whole slide images of hematoxylin and eosin stained tumors. A virtual slide is a high-definition, fully digital capture of pathological specimen which can be used for pathological evaluation without significant loss of fidelity, scanned at a resolution of 0.25 μm per pixel. Tumor regions are digitally annotated on each virtual slide by a pathologist using digital pathology analysis platform QuPath. A tumor tile is a virtual tile sub-image focused only on areas of tumor, and with reduced focal depth to limit file size and promote processing. The deep learning pipeline automatically and efficiently creates tumor tiles for each tumor region based on a specified tile size.

A neural network optimized to pathology imaging is then used to extract features from tumor tiles. During this computationally-intensive process, the neural network “learns” combinations of histology features most characteristic of a specified objective (in this case which cancers are most likely to recur). To accomplish this task, pixel data from extracted image tiles were normalized and then trained via a Tensorflow/Keras implementation of the Xception DCNN model, with weights initialized using pretraining.

In order to reduce bias against sparse categories, training batches were filled with tiles in a manner that was balanced according to the output category. At the time of training, tiles were randomly vertically and horizontally flipped, as well as randomly rotated 90, 180, or 270 degrees. Training performance was evaluated on a validation dataset chosen at the time of training using K-fold cross-validation, averaged across the folds. Using the algorithm on pilot data, an average cross-validated AUC of 0.91 was achieved, which is early evidence of strong predictive performance.

Accordingly, a first example embodiment may involve may involve generating tumor image tiles from images of HPV+ HNSCC tumors, wherein the tumor image tiles are respectively labelled with indicators of tumor recurrence. These labels may indicate whether the tumor image tiles depict recurrent or non-recurrent tumors, for example. The first example embodiment may further involve training a neural network with the tumor image tiles as labelled, wherein the training results in the neural network learning combinations of histology features characteristic of tumor recurrence.

A second example embodiment may involve obtaining tumor image tiles from images of HPV+ HNSCC tumors. The second example embodiment may further involve providing the tumor image tiles to a trained neural network, wherein the neural network was trained to identify combinations of histology features characteristic of tumor recurrence and to generate classifications of the tumor image tiles based on likelihood of tumor recurrence. The second example embodiment may further involve storing the classifications as respectively associated with their corresponding tumor image tiles.

The first and second example embodiments may be combined with one another in various ways and/or implemented in various types of computing devices by instructions stored on computer-readable media.

These as well as other embodiments, aspects, advantages, and alternatives will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

FIG. 1 illustrates a schematic drawing of a computing device, in accordance with example embodiments.

FIG. 2 illustrates a schematic drawing of a server device cluster, in accordance with example embodiments.

FIG. 3 depict an ANN architecture, in accordance with example embodiments.

FIGS. 4A and 4B depict training an ANN, in accordance with example embodiments.

FIG. 5A depicts a CNN architecture, in accordance with example embodiments.

FIG. 5B depicts a convolution, in accordance with example embodiments.

FIG. 6 depicts three case-control cohorts, in accordance with example embodiments.

FIG. 7 is a flow chart, in accordance with example embodiments.

FIG. 8 is a flow chart, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein.

Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

I. Example Computing Devices and Cloud-Based Computing Environments

The following embodiments describe architectural and operational aspects of example computing devices and systems that may employ the disclosed ANN and DCNN implementations, as well as the features and advantages thereof.

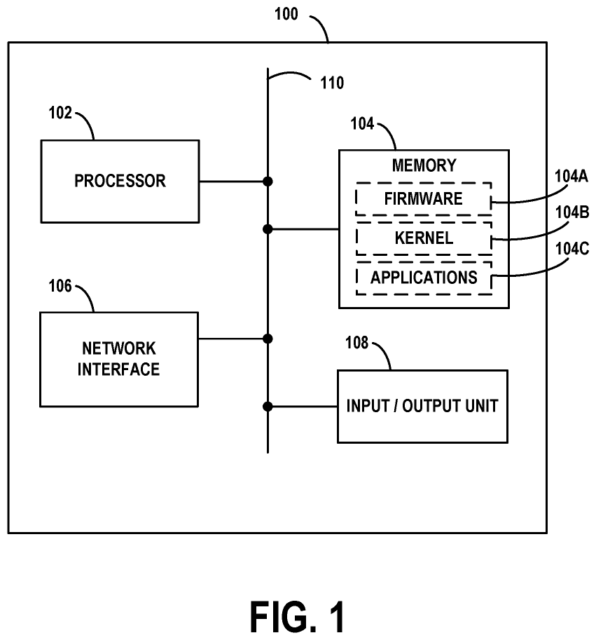

FIG. 1 is a simplified block diagram exemplifying a computing device 100, illustrating some of the components that could be included in a computing device arranged to operate in accordance with the embodiments herein. Computing device 100 could be a client device (e.g., a device actively operated by a user), a server device (e.g., a device that provides computational services to client devices), or some other type of computational platform. Some server devices may operate as client devices from time to time in order to perform particular operations, and some client devices may incorporate server features.

In this example, computing device 100 includes processor 102, memory 104, network interface 106, and an input/output unit 108, all of which may be coupled by a system bus 110 or a similar mechanism. In some embodiments, computing device 100 may include other components and/or peripheral devices (e.g., detachable storage, printers, and so on).

Processor 102 may be one or more of any type of computer processing element, such as a central processing unit (CPU), a co-processor (e.g., a mathematics, graphics, or encryption co-processor), a digital signal processor (DSP), a network processor, and/or a form of integrated circuit or controller that performs processor operations. In some cases, processor 102 may be one or more single-core processors. In other cases, processor 102 may be one or more multi-core processors with multiple independent processing units. Processor 102 may also include register memory for temporarily storing instructions being executed and related data, as well as cache memory for temporarily storing recently-used instructions and data.

Memory 104 may be any form of computer-usable memory, including but not limited to random access memory (RAM), read-only memory (ROM), and non-volatile memory. This may include flash memory, hard disk drives, solid state drives, re-writable compact discs (CDs), re-writable digital video discs (DVDs), and/or tape storage, as just a few examples. Computing device 100 may include fixed memory as well as one or more removable memory units, the latter including but not limited to various types of secure digital (SD) cards. Thus, memory 104 represents both main memory units, as well as long-term storage. Other types of memory may include biological memory.

Memory 104 may store program instructions and/or data on which program instructions may operate. By way of example, memory 104 may store these program instructions on a non-transitory, computer-readable medium, such that the instructions are executable by processor 102 to carry out any of the methods, processes, or operations disclosed in this specification or the accompanying drawings.

As shown in FIG. 1, memory 104 may include firmware 104A, kernel 104B, and/or applications 104C. Firmware 104A may be program code used to boot or otherwise initiate some or all of computing device 100. Kernel 104B may be an operating system, including modules for memory management, scheduling and management of processes, input/output, and communication. Kernel 104B may also include device drivers that allow the operating system to communicate with the hardware modules (e.g., memory units, networking interfaces, ports, and busses), of computing device 100. Applications 104C may be one or more user-space software programs, such as web browsers or email clients, as well as any software libraries used by these programs. Memory 104 may also store data used by these and other programs and applications.

Network interface 106 may take the form of one or more wireline interfaces, such as Ethernet (e.g., Fast Ethernet, Gigabit Ethernet, and so on). Network interface 106 may also support communication over one or more non-Ethernet media, such as coaxial cables or power lines, or over wide-area media, such as Synchronous Optical Networking (SONET) or digital subscriber line (DSL) technologies. Network interface 106 may additionally take the form of one or more wireless interfaces, such as IEEE 802.11 (Wifi), BLUETOOTH®, global positioning system (GPS), or a wide-area wireless interface. However, other forms of physical layer interfaces and other types of standard or proprietary communication protocols may be used over network interface 106. Furthermore, network interface 106 may comprise multiple physical interfaces. For instance, some embodiments of computing device 100 may include Ethernet, BLUETOOTH®, and Wifi interfaces.

Input/output unit 108 may facilitate user and peripheral device interaction with example computing device 100. Input/output unit 108 may include one or more types of input devices, such as a keyboard, a mouse, a touch screen, and so on. Similarly, input/output unit 108 may include one or more types of output devices, such as a screen, monitor, printer, and/or one or more light emitting diodes (LEDs). Additionally or alternatively, computing device 100 may communicate with other devices using a universal serial bus (USB) or high-definition multimedia interface (HDMI) port interface, for example.

In some embodiments, one or more instances of computing device 100 may be deployed to support a clustered architecture. The exact physical location, connectivity, and configuration of these computing devices may be unknown and/or unimportant to client devices. Accordingly, the computing devices may be referred to as “cloud-based” devices that may be housed at various remote data center locations.

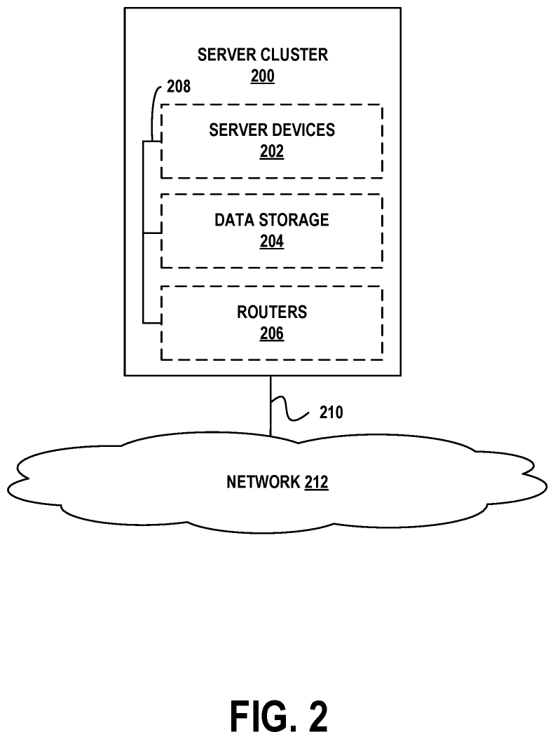

FIG. 2 depicts a cloud-based server cluster 200 in accordance with example embodiments. In FIG. 2, operations of a computing device (e.g., computing device 100) may be distributed between server devices 202, data storage 204, and routers 206, all of which may be connected by local cluster network 208. The number of server devices 202, data storages 204, and routers 206 in server cluster 200 may depend on the computing task(s) and/or applications assigned to server cluster 200.

For example, server devices 202 can be configured to perform various computing tasks of computing device 100. Thus, computing tasks can be distributed among one or more of server devices 202. To the extent that these computing tasks can be performed in parallel, such a distribution of tasks may reduce the total time to complete these tasks and return a result. For purpose of simplicity, both server cluster 200 and individual server devices 202 may be referred to as a “server device.” This nomenclature should be understood to imply that one or more distinct server devices, data storage devices, and cluster routers may be involved in server device operations.

Data storage 204 may be data storage arrays that include drive array controllers configured to manage read and write access to groups of hard disk drives and/or solid state drives. The drive array controllers, alone or in conjunction with server devices 202, may also be configured to manage backup or redundant copies of the data stored in data storage 204 to protect against drive failures or other types of failures that prevent one or more of server devices 202 from accessing units of cluster data storage 204. Other types of memory aside from drives may be used.

Routers 206 may include networking equipment configured to provide internal and external communications for server cluster 200. For example, routers 206 may include one or more packet-switching and/or routing devices (including switches and/or gateways) configured to provide (i) network communications between server devices 202 and data storage 204 via cluster network 208, and/or (ii) network communications between the server cluster 200 and other devices via communication link 210 to network 212.

Additionally, the configuration of cluster routers 206 can be based at least in part on the data communication requirements of server devices 202 and data storage 204, the latency and throughput of the local cluster network 208, the latency, throughput, and cost of communication link 210, and/or other factors that may contribute to the cost, speed, fault-tolerance, resiliency, efficiency and/or other design goals of the system architecture.

As a possible example, data storage 204 may include any form of database, such as a structured query language (SQL) database. Various types of data structures may store the information in such a database, including but not limited to tables, arrays, lists, trees, and tuples. Furthermore, any databases in data storage 204 may be monolithic or distributed across multiple physical devices.

Server devices 202 may be configured to transmit data to and receive data from cluster data storage 204. This transmission and retrieval may take the form of SQL queries or other types of database queries, and the output of such queries, respectively. Additional text, images, video, and/or audio may be included as well. Furthermore, server devices 202 may organize the received data into web page representations. Such a representation may take the form of a markup language, such as the hypertext markup language (HTML), the extensible markup language (XML), or some other standardized or proprietary format. Moreover, server devices 202 may have the capability of executing various types of computerized scripting languages, such as but not limited to Perl, Python, PHP Hypertext Preprocessor (PHP), Active Server Pages (ASP), JavaScript, and so on. Computer program code written in these languages may facilitate the providing of web pages to client devices, as well as client device interaction with the web pages.

II. Artificial Neural Networks

An ANN is a computational model in which a number of simple units, working individually in parallel and without central control, combine to solve complex problems. While this model may resemble an animal's brain in some respects, analogies between ANNs and brains are tenuous at best. Modern ANNs have a fixed structure, a deterministic mathematical learning process, are trained to solve one problem at a time, and are much smaller than their biological counterparts.

A. Example ANN

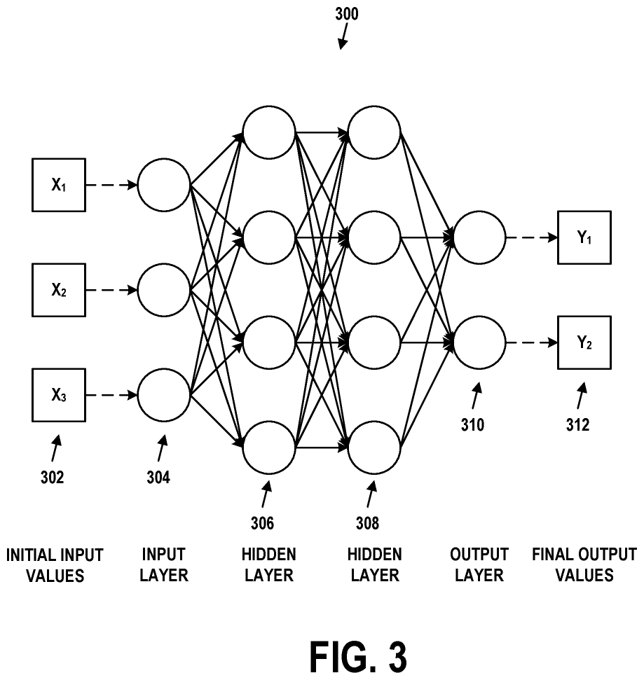

An ANN is represented as a number of nodes that are arranged into a number of layers, with connections between the nodes of adjacent layers. An example ANN 300 is shown in FIG. 3. ANN 300 represents a feed-forward multilayer neural network, but similar structures and principles are used in CNNs, recurrent neural networks, and recursive neural networks, for example.

Regardless, ANN 300 consists of four layers: input layer 304, hidden layer 306, hidden layer 308, and output layer 310. The three nodes of input layer 304 respectively receive X₁, X₂, and X₃ from initial input values 302. The two nodes of output layer 310 respectively produce Y₁ and Y₂ for final output values 312. ANN 300 is a fully-connected network, in that nodes of each layer aside from input layer 304 receive input from all nodes in the previous layer.

The solid arrows between pairs of nodes represent connections through which intermediate values flow, and are each associated with a respective weight that is applied to the respective intermediate value. Each node performs an operation on its input values and their associated weights (e.g., values between 0 and 1, inclusive) to produce an output value. In some cases this operation may involve a dot-product sum of the products of each input value and associated weight. An activation function may be applied to the result of the dot-product sum to produce the output value. Other operations are possible.

For example, if a node receives input values {x₁, x₂, . . . , xₙ} on n connections with respective weights of {w₁, w₂, . . . , wₙ} , the dot-product sum d may be determined as:

$$d = {\sum }_{i = 1}^{n} {x}_{i} {w}_{i} + b \qquad ( 1 )$$

Where b is a node-specific or layer-specific bias.

Notably, the fully-connected nature of ANN 300 can be used to effectively represent a partially-connected ANN by giving one or more weights a value of 0. Similarly, the bias can also be set to 0 to eliminate the b term.

An activation function, such as the logistic function, may be used to map d to an output value y that is between 0 and 1, inclusive:

$$y = \frac{1}{1 + {e}^{- d}} \qquad ( 2 )$$

Functions other than the logistic function, such as the sigmoid or tanh functions, may be used instead.

Then, y may be used on each of the node's output connections, and will be modified by the respective weights thereof. Particularly, in ANN 300, input values and weights are applied to the nodes of each layer, from left to right until final output values 312 are produced. If ANN 300 has been fully trained, final output values 312 are a proposed solution to the problem that ANN 300 has been trained to solve. In order to obtain a meaningful, useful, and reasonably accurate solution, ANN 300 requires at least some extent of training.

B. Training

Training an ANN usually involves providing the ANN with some form of supervisory training data, namely sets of input values and desired, or ground truth, output values. For ANN 300, this training data may include m sets of input values paired with output values. More formally, the training data may be represented as:

{X_{1,i}, X_{2,i}, X_{3,i}, [symbol] , [symbol] } (3)

Where i=1 . . . m, and [symbol] and [symbol] are the desired output values for the input values of X_{1,i}, X_{2,i}, and X_{3,i}.

The training process involves applying the input values from such a set to ANN 300 and producing associated output values. A loss function is used to evaluate the error between the produced output values and the ground truth output values. This loss function may be a sum of differences, mean squared error, or some other metric. In some cases, error values are determined for all of the m sets, and the error function involves calculating an aggregate (e.g., an average) of these values.

Once the error is determined, the weights on the connections are updated in an attempt to reduce the error. In simple terms, this update process should reward “good” weights and penalize “bad” weights. Thus, the updating should distribute the “blame” for the error through ANN 300 in a fashion that results in a lower error for future iterations of the training data.

The training process continues applying the training data to ANN 300 until the weights converge. Convergence occurs when the error is less than a threshold value or the change in the error is sufficiently small between consecutive iterations of training. At this point, ANN 300 is said to be “trained” and can be applied to new sets of input values in order to predict output values that are unknown.

Most training techniques for ANNs make use of some form of backpropagation. Backpropagation distributes the error one layer at a time, from right to left, through ANN 300. Thus, the weights of the connections between hidden layer 308 and output layer 310 are updated first, the weights of the connections between hidden layer 306 and hidden layer 308 are updated second, and so on. This updating is based on the derivative of the activation function.

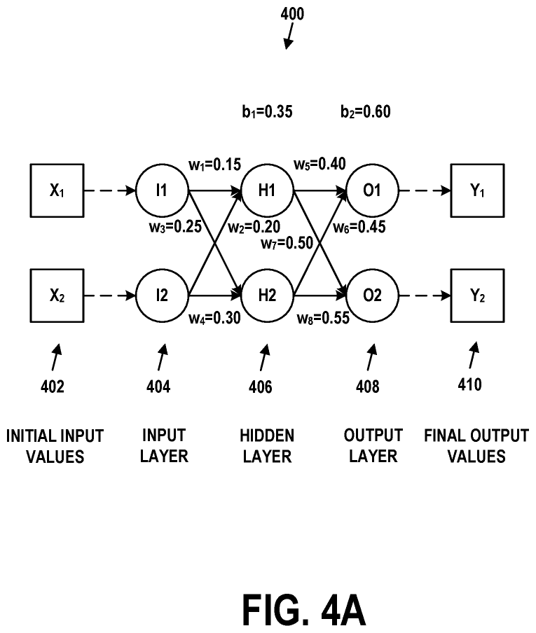

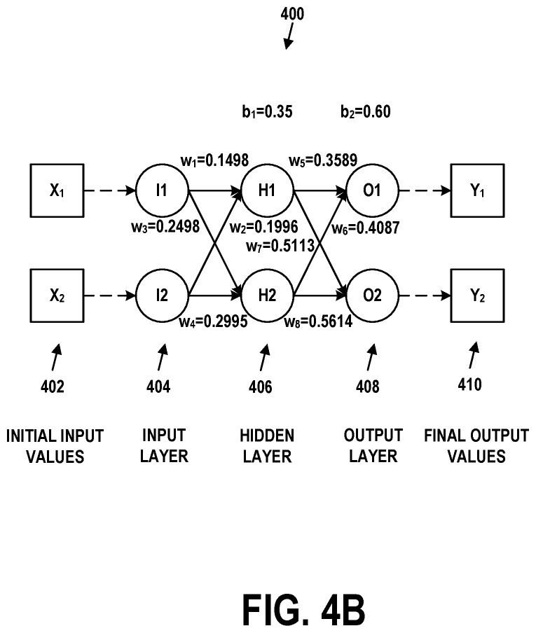

In order to further explain error determination and backpropagation, it is helpful to look at an example of the process in action. However, backpropagation becomes quite complex to represent except on the simplest of ANNs. Therefore, FIG. 4A introduces a very simple ANN 400 in order to provide an illustrative example of backpropagation.

TABLE 1

| Weight | Nodes |
|---|---|
| w₁ | I1, H1 |
| w₂ | I2, H1 |
| w₃ | I1, H2 |
| w₄ | I2, H2 |
| w₅ | H1, O1 |
| w₆ | H2, O1 |
| w₇ | H1, O2 |
| w₈ | H2, O2 |

ANN 400 consists of three layers, input layer 404, hidden layer 406, and output layer 408, each having two nodes. Initial input values 402 are provided to input layer 404, and output layer 408 produces final output values 410. Weights have been assigned to each of the connections. Also, bias b₁=0.35 is applied to the net input of each node in hidden layer 406, and a bias b₂=0.60 is applied to the net input of each node in output layer 408. For clarity, Table I maps weights to pair of nodes with connections to which these weights apply. As an example, w₂ is applied to the connection between nodes 12 and HI, w₇ is applied to the connection between nodes H1 and O2, and so on.

For purposes of demonstration, initial input values are set to X₁=0.05 and X₂=0.10, and the desired output values are set to [symbol] =0.01 and [symbol] =0.99. Thus, the goal of training ANN 400 is to update the weights over some number of feed forward and backpropagation iterations until the final output values 410 are sufficiently close to [symbol] =0.01 and [symbol] =0.99 when X₁=0.05 and X₂=0.10. Note that use of a single set of training data effectively trains ANN 400 for just that set. If multiple sets of training data are used, ANN 400 will be trained in accordance with those sets as well.

1. Example Feed Forward Pass

To initiate the feed forward pass, net inputs to each of the nodes in hidden layer 406 are calculated. From the net inputs, the outputs of these nodes can be found by applying the activation function.

For node H1, the net input net_{H1} is:

$$\begin{matrix}{net}_{H 1} = {w}_{1} {X}_{1} + {w}_{2} {X}_{2} + {b}_{1} \\ = ( 0.15 ) ( 0.05 ) + ( 0.2 ) ( 0.1 ) + 0.35 = 0.3775\end{matrix} \qquad ( 4 )$$

Applying the activation function (here, the logistic function) to this input determines that the output of node H1, out_{H1} is:

$$\begin{matrix}{out}_{H 1} = \frac{1}{1 + {e}^{- {net}_{H 1}}} \\ = 0.593269992\end{matrix} \qquad ( 5 )$$

Following the same procedure for node H2, the output out_{H2} is 0.596884378. The next step in the feed forward iteration is to perform the same calculations for the nodes of output layer 408. For example, net input to node O1, net₀₁ is:

$$\begin{matrix}{net}_{O 1} = {w}_{5} {out}_{H 1} + {w}_{6} {out}_{H 2} + {b}_{2} \\ = ( 0.4 ) ( 0.593269992 ) + ( 0.45 ) ( 0.596884378 ) + 0.6 \\ = 1.105905967\end{matrix} \qquad ( 6 )$$

Thus, output for node 01, out₀₁ is:

$$\begin{matrix}{out}_{O 1} = \frac{1}{1 + {e}^{- {net}_{O 1}}} \\ = 0.75136507\end{matrix} \qquad ( 7 )$$

Following the same procedure for node O2. the output out₀₂ is 0.772928465.

At this point, the total error, Δ, can be determined based on a loss function. In this case, the loss function can be the sum of the squared error for the nodes in output layer 408. In other words:

$$\begin{matrix}Δ = {Δ}_{O 1} {Δ}_{O 2} \\ = \frac{1}{2} {( - 0.75136507 - 0.01 )}^{2} + \frac{1}{2} {( 0.772928465 - 0.99 )}^{2} \\ = 0.274811083 + 0.023560026 = 0.298371109\end{matrix} \qquad ( 8 )$$

The multiplicative constant ½ in each term is used to simplify differentiation during backpropagation. Since the overall result is scaled by a learning rate anyway, this constant does not negatively impact the training. Regardless, at this point, the feed forward iteration completes and backpropagation begins.

2. Backpropagation

As noted above, a goal of backpropagation is to use Δ to update the weights so that they contribute less error in future feed forward iterations. As an example, consider the weight w₅. The goal involves determining how much the change in w₅ affects Δ. This can be expressed as the partial derivative

$$\frac{\partial Δ}{\partial {w}_{5}} .$$

Using the chain rule, this term can be expanded as:

$$\frac{\partial Δ}{\partial {w}_{5}} = \frac{\partial Δ}{\partial {out}_{O 1}} \times \frac{\partial {out}_{O 1}}{\partial {net}_{O 1}} \times \frac{\partial {net}_{O 1}}{\partial {w}_{5}} \qquad ( 9 )$$

Thus, the effect on Δ of change to w₅ is equivalent to the product of (i) the effect on Δ of change to out_{O1}, (ii) the effect on out_{O1} of change to net_{O1}, and (iii) the effect on neto1 of change to w₅. Each of these multiplicative terms can be determined independently. Intuitively, this process can be thought of as isolating the impact of w₅ on net_{O1}, the impact of net_{O1} on out_{O1}, and the impact of out_{O1} on Δ.

Starting with

$$\frac{\partial Δ}{\partial {out}_{O 1}} ,$$

the expression for Δ is:

Δ=½)out_{O1}− [symbol] )²+½(out_{O2}− [symbol] )² (10)

When taking the partial derivative with respect to out_{O1}, the term containing out_{O2} is effectively a constant because changes to out_{O1} do not affect this term. Therefore:

$$\begin{matrix}\frac{\partial Δ}{\partial {out}_{O 1}} = {out}_{O 1} - {\hat{Y}}_{1} \\ = 0.75136507 - 0.01 = 0.74136507\end{matrix} \qquad ( 11 )$$

For

$$\frac{\partial {out}_{O 1}}{\partial {net}_{O 1}} ,$$

the expression for out_{O1}, Equation 5, is:

$${out}_{O 1} = \frac{1}{1 + {e}^{- {net}_{O 1}}} \qquad ( 12 )$$

Therefore, taking the derivative of the logistic function:

$$\partial {out}_{O 1} = 0.75136507 ( 1 - 0.75136507 ) = 0.186815602 \qquad ( 13 )$$

For

$$\frac{\partial {net}_{O 1}}{\partial {w}_{5}} ,$$

the expression for net_{O1}, from Equation 6, is:

net_{O1}=w₅out_{H1}+w₆out_{H2}+b₂ (14)

Similar to the expression for Δ, taking the derivative of this expression involves treating the two rightmost terms as constants, since w₅ does not appear in those terms. Thus:

$$\frac{\partial {net}_{O 1}}{\partial {w}_{5}} = {out}_{H 1} = 0.593269992 \qquad ( 15 )$$

These three partial derivative terms can be put together to solve Equation 9:

$$\begin{matrix}\frac{\partial Δ}{\partial {w}_{5}} = ( {out}_{O 1} - {\hat{Y}}_{1} ) {out}_{O 1} ( 1 - {out}_{O 1} ) {out}_{H 1} \\ = ( 0.74136507 ) ( 0.186815602 ) ( 0.593269992 ) = 0.082167041\end{matrix} \qquad ( 16 )$$

Then, this value can be subtracted from w₅. Often a gain, 0<α≤1, is applied to

$$\frac{\partial Δ}{\partial {w}_{5}}$$

to control how aggressively the ANN responds to errors. Assuming that α=0.5, the full expression is:

$$\begin{matrix}{w}_{5} = {w}_{5} - α \frac{\partial Δ}{\partial {w}_{5}} \\ = 0.4 - ( 0.5 ) ( 0.082167041 ) = 0.35891648\end{matrix} \qquad ( 17 )$$

This process can be repeated for the other weights feeding into output layer 408. The results are:

w₆=0.408666186 (18)

w₇=0.511301270

w₈=0.561370121

Note that no weights are updated until the updates to all weights have been determined at the end of backpropagation. Then, all weights are updated before the next feed forward iteration.

Next, updates to the remaining weights, w₁, w₂, w₃, and w₄ are calculated. This involves continuing the backpropagation pass to hidden layer 406. Considering w₁ and using a similar derivation as above:

$$\frac{\partial Δ}{\partial {w}_{1}} = \frac{\partial Δ}{\partial {out}_{H 1}} \times \frac{\partial {out}_{H 1}}{\partial {net}_{H 1}} \times \frac{\partial {net}_{H 1}}{\partial {w}_{1}} \qquad ( 19 )$$

One difference, however, between the backpropagation techniques for output layer 408 and hidden layer 406 is that each node in hidden layer 406 contributes to the error of all nodes in output layer 408. Therefore:

$$\\ \begin{matrix}\frac{\partial Δ}{\partial {out}_{H 1}} = \frac{\partial {Δ}_{O 1}}{\partial {out}_{H 1}} + \frac{\partial {Δ}_{O 2}}{\partial {out}_{H 1}} & ( 20 )\end{matrix}$$

Beginning with

$$\\ \begin{matrix}\frac{\partial {Δ}_{O 1}}{\partial {out}_{H 1}} : \frac{\partial {Δ}_{O 1}}{\partial {out}_{H 1}} = \frac{\partial {Δ}_{O 1}}{\partial {net}_{O 1}} \times \frac{\partial {net}_{O 1}}{\partial {out}_{H 1}} & ( 21 )\end{matrix}$$

Regarding

$$\\ \frac{\partial {Δ}_{O 1}}{\partial {net}_{O 1}} ,$$

the impact of change in net_{O1} on Δ_{O1} is the same as impact of change in net_{O1} on Δ, so the calculations performed above for Equations 11 and 13 can be reused:

$$\\ \begin{matrix}\begin{matrix}\frac{\partial {Δ}_{O 1}}{\partial {net}_{O 1}} = \frac{\partial Δ}{\partial {out}_{O 1}} \times \frac{\partial {out}_{O 1}}{\partial {net}_{O 1}} \\ = ( 0.74136507 ) ( 0.186815602 ) = 0.138498562\end{matrix} & ( 22 )\end{matrix}$$

Regarding

$$\\ \frac{\partial {net}_{O 1}}{\partial {out}_{H 1}} ,$$

net_{O1} can be expressed as:

net_{O1}=w₅out_{H1}+w₆out_{H2}+b₂ (23)

Thus:

$$\\ \begin{matrix}\frac{\partial {net}_{O 1}}{\partial {out}_{H 1}} = {w}_{5} = 0.4 & ( 24 )\end{matrix}$$

Therefore, Equation 21 can be solved as:

$$\\ \begin{matrix}\begin{matrix}\frac{\partial {Δ}_{O 1}}{\partial {out}_{H 1}} = \frac{\partial {Δ}_{O 1}}{\partial {net}_{O 1}} \times \frac{\partial {net}_{O 1}}{\partial {out}_{H 1}} \\ = ( 0.138498562 ) ( 0.4 ) = 0.055399425\end{matrix} & ( 25 )\end{matrix}$$

Following a similar procedure for

$$\\ \frac{\partial {Δ}_{O 2}}{\partial {out}_{H 1}}$$

results in:

$$\\ \begin{matrix}\frac{\partial {Δ}_{O 2}}{\partial {out}_{H 1}} = - 0.019049119 & ( 26 )\end{matrix}$$

Consequently, Equation 20 can be solved as:

$$\\ \begin{matrix}\begin{matrix}\frac{\partial Δ}{\partial {out}_{H 1}} = \frac{\partial {Δ}_{O 1}}{\partial {out}_{H 1}} + \frac{\partial {Δ}_{O 2}}{\partial {out}_{H 1}} \\ = 0.055399425 - 0.019049119 = 0.036350306\end{matrix} & ( 27 )\end{matrix}$$

This also solves for the first term of Equation 19. Next, since node H1 uses the logistic function as its activation function to relate out_{H1} and net_{H1}, the second term of Equation 19,

$$\\ \frac{\partial {out}_{H 1}}{\partial {net}_{H 1}} ,$$

can be determined as:

$$\\ \begin{matrix}\partial {out}_{H 1} = 0.59326999 ( 1 - 0.59326999 ) = 0.241300709 & ( 28 )\end{matrix}$$

Then, as net_{H1} can be expressed as:

net_{H1}=w₁X₁+w₂X₂+b₁ (29)

Thus, the third term of Equation 19 is:

$$\\ \begin{matrix}\frac{\partial {net}_{H 1}}{\partial {w}_{1}} = {X}_{1} = 0.05 & ( 30 )\end{matrix}$$

Putting the three terms of Equation 19 together, the result is:

$$\\ \begin{matrix}\begin{matrix}\frac{\partial Δ}{\partial {w}_{1}} = \frac{\partial Δ}{\partial {out}_{H 1}} \times \frac{\partial {out}_{H 1}}{\partial {net}_{H 1}} \times \frac{\partial {net}_{H 1}}{\partial {w}_{1}} \\ = ( 0.036350306 ) ( 0.241300709 ) ( 0.05 ) = 0.000438568\end{matrix} & ( 31 )\end{matrix}$$

With this, w₁ can be updated as:

$$\\ \begin{matrix}\begin{matrix}{w}_{1} = {w}_{1} - α \frac{\partial Δ}{\partial {w}_{1}} \\ = 0.15 - ( 0.5 ) ( 0.000438568 ) = 0.149780716\end{matrix} & ( 32 )\end{matrix}$$

This process can be repeated for the other weights feeding into hidden layer 406. The results are:

w₂=0.19956143 (33)

w₃=0.24975114

w₄=0.29950229

At this point, the backpropagation iteration is over, and all weights have been updated. FIG. 4B shows ANN 400 with these updated weights, values of which are rounded to four decimal places for sake of convenience. ANN 400 may continue to be trained through subsequent feed forward and backpropagation iterations. For instance, the iteration carried out above reduces the total error, Δ, from 0.298371109 to 0.291027924. While this may seem like a small improvement, over several thousand feed forward and backpropagation iterations the error can be reduced to less than 0.0001. At that point, the values of Y₁ and Y₂ will be close to the target values of 0.01 and 0.99, respectively.

In some cases, an equivalent amount of training can be accomplished with fewer iterations if the hyperparameters of the system (e.g., the biases b₁ and b₂ and the learning rate α) are adjusted. For instance, the setting the learning rate closer to 1.0 may result in the error rate being reduced more rapidly. Additionally, the biases can be updated as part of the learning process in a similar fashion to how the weights are updated.

Regardless, ANN 400 is just a simplified example. Arbitrarily complex ANNs can be developed with the number of nodes in each of the input and output layers tuned to address specific problems or goals. Further, more than one hidden layer can be used and any number of nodes can be in each hidden layer.

C. Convolutional Neural Networks

CNNs are similar to ANNs, in that they consist of some number of layers of nodes, with weighted connections therebetween and possible per-layer biases. The weights and biases may be updated by way of feed forward and backpropagation procedures discussed above. A loss function may be used to compare output values of feed forward processing to desired output values.

On the other hand, CNNs are usually designed with the explicit assumption that the initial input values are derived from one or more images. In some embodiments, each color channel of each pixel in an image patch is a separate initial input value. Assuming three color channels per pixel (e.g., red, green, and blue), even a small 32×32 patch of pixels will result in 3072 incoming weights for each node in the first hidden layer. Clearly, using a naïve ANN for image processing could lead to a very large and complex model that would take long to train.

Instead, CNNs are designed to take advantage of the inherent structure that is found in almost all images. In particular, nodes in a CNN are only connected to a small number of nodes in the previous layer. This CNN architecture can be thought of as three dimensional, with nodes arranged in a block with a width, a height, and a depth. For example, the aforementioned 32×32 patch of pixels with 3 color channels may be arranged into an input layer with a width of 32 nodes, a height of 32 nodes, and a depth of 3 nodes.

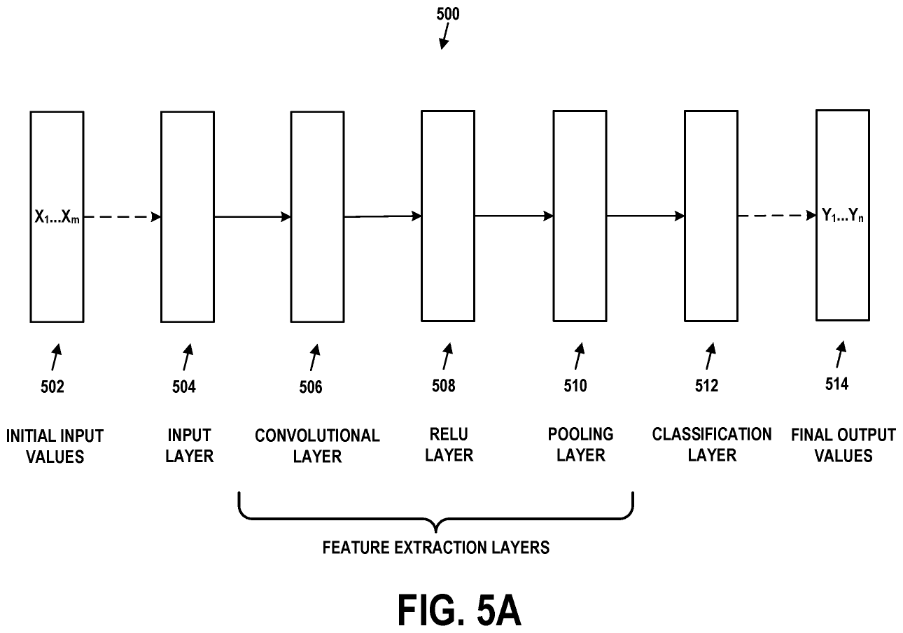

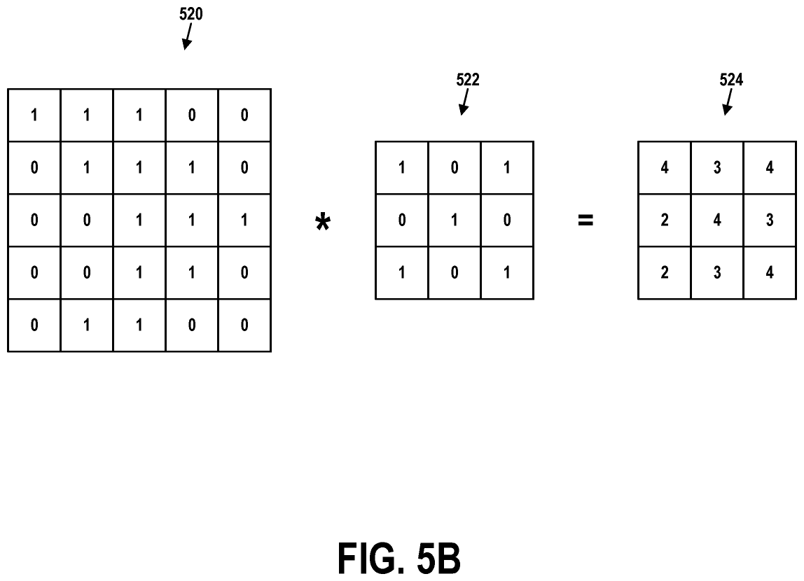

An example CNN 500 is shown in FIG. 5A. Initial input values 502, represented as pixels X₁ . . . Xₘ, are provided to input layer 504. As discussed above, input layer 504 may have three dimensions based on the width, height, and number of color channels of pixels X₁ . . . Xₘ. Input layer 504 provides values into one or more sets of feature extraction layers, each set containing an instance of convolutional layer 506, RELU layer 508, and pooling layer 510. The output of pooling layer 510 is provided to one or more classification layers 512. Final output values 514 may be arranged in a feature vector representing a concise characterization of initial input values 502.

Convolutional layer 506 may transform its input values by sliding one or more filters around the three-dimensional spatial arrangement of these input values. A filter is represented by biases applied to the nodes and the weights of the connections therebetween, and generally has a width and height less than that of the input values. The result for each filter may be a two-dimensional block of output values (referred to as an feature map) in which the width and height can have the same size as those of the input values, or one or more of these dimensions may have different size. The combination of each filter's output results in layers of feature maps in the depth dimension, in which each layer represents the output of one of the filters.

Applying the filter may involve calculating the dot-product sum between the entries in the filter and a two-dimensional depth slice of the input values. An example of this is shown in FIG. 5B. Matrix 520 represents input to a convolutional layer, and thus could be image data, for example. The convolution operation overlays filter 522 on matrix 520 to determine output 524. For instance, when filter 522 is positioned in the top left corner of matrix 520, and the dot-product sum for each entry is calculated, the result is 4. This is placed in the top left corner of output 524.

Turning back to FIG. 5A, a CNN learns filters during training such that these filters can eventually identify certain types of features at particular locations in the input values. As an example, convolutional layer 506 may include a filter that is eventually capable of detecting edges and/or colors in the image patch from which initial input values 502 were derived. A hyperparameter called receptive field determines the number of connections between each node in convolutional layer 506 and input layer 504. This allows each node to focus on a subset of the input values.

RELU layer 508 applies an activation function to output provided by convolutional layer 506. In practice, it has been determined that the rectified linear unit (RELU) function, or a variation thereof, appears to provide the best results in CNNs. The RELU function is a simple thresholding function defined as f(x)=max (0, x). Thus, the output is 0 when x is negative, and x when x is non-negative. A smoothed, differentiable approximation to the RELU function is the softplus function. It is defined as f(x)=log (1+e^{x}). Nonetheless, other functions may be used in this layer.

Pooling layer 510 reduces the spatial size of the data by downsampling each two-dimensional depth slice of output from RELU layer 508. One possible approach is to apply a 2×2 filter with a stride of 2 to each 2×2 block of the depth slices. This will reduce the width and height of each depth slice by a factor of 2, thus reducing the overall size of the data by 75%.

Classification layer 512 computes final output values 514 in the form of a feature vector. As an example, in a CNN trained to be an image classifier, each entry in the feature vector may encode a probability that the image patch contains a particular class of item (e.g., a particular type of tumor or indication of a tumor feature, etc.).

In some embodiments, there are multiple sets of the feature extraction layers. Thus, an instance of pooling layer 510 may provide output to an instance of convolutional layer 506. Further, there may be multiple instances of convolutional layer 506 and RELU layer 508 for each instance of pooling layer 510.

CNN 500 represents a general structure that can be used in image processing. Convolutional layer 506 and classification layer 512 apply weights and biases similarly to layers in ANN 3400, and these weights and biases may be updated during backpropagation so that CNN 500 can learn. On the other hand, RELU layer 508 and pooling layer 510 generally apply fixed operations and thus might not learn.

Not unlike an ANN, a CNN can include a different number of layers than is shown in the examples herein, and each of these layers may include a different number of nodes. Thus, CNN 500 is merely for illustrative purposes and should not be considered to limit the structure of a CNN.

III. Machine Learning Based Recurrence Prediction for HPV+ HNSCCs

As noted above, since HPV+HNSCCs are often cured, a therapeutic objective has been to reduce morbidity caused by their treatment with high dose radiation plus cisplatin, which often leaves life-long disabilities. Unfortunately, attempts to decrease radiation dose or supplant cisplatin use with anti-EGFR therapy have reduced survival in some trials because of a lack of adequate biomarkers to identify recurrence-prone patients. Prospective identification of such patients would exclude them from therapy de-escalation and select them for testing of novel therapies. Likewise, accurately identifying patients at lowest recurrence risk would reduce treatment-related morbidity by allowing aggressive therapy de-escalation in suitable patients.

A prior trend away from surgery for oropharyngeal cancer was reversed by recent advances that reduce surgical morbidity by allowing minimally invasive access to the oropharynx. Specifically, FDA approval of TORS (transoral robotic surgery) for oropharyngeal tumors in 2009 has led to rapid and ongoing adoption of this modality, making HPV+ HNSCC cohorts previously used to assess adverse molecular features less representative of modern therapeutic practice. TORS has been associated with better survival for oropharyngeal cancers relative to other surgical methods and has facilitated trials evaluating reduced postoperative radiation and/or elimination of cytotoxic chemotherapy for cases thought to have low risk of recurrence. However, surgical trials continue to risk-stratify patients using clinical and pathologic criteria that were developed from older cohorts where non-TORS based-therapy predominated and have modest utility under the modern TORS treatment paradigm. Developing machine learning-based histologic criteria to distinguish HPV+HNSCCs with high lethal potential after TORS would facilitate testing of novel adjuvant approaches for them and greater therapy de-escalation for typical cases.

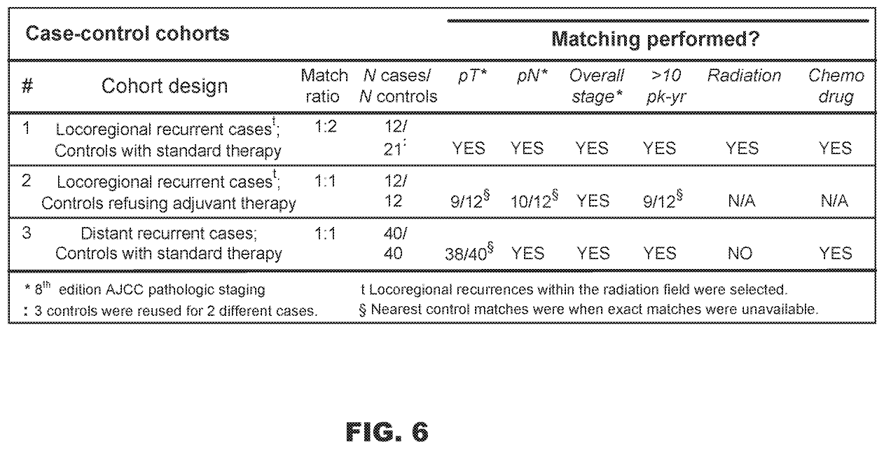

To facilitate machine learning-based prospective identification of patients at risk of recurrence post-TORS, a case-control cohort was developed from 634 treatment-naïve HPV+ HNSCC cases in the oropharynx consecutively managed during 2007-2017 with primary TORS plus neck dissection at the University of Pennsylvania. For all locoregional recurrences (LRRs) identified, radiation plans were reviewed to exclude cases where recurrence arose outside the surgical or radiation field (receiving dose <50 Gy). This process identified a total of 12 LRRs that reflect a maximally therapy-resistant phenotype for molecular analysis. In addition, 40 cases of distant metastatic recurrence (DMR) were identified, and all were retained irrespective of whether they received chemotherapy, because standard systemic therapy (cisplatin) does not reduce distant failure for this disease. To confirm that pulmonary distant metastases were truly of HNSCC origin and not from a p16+ squamous non-small cell lung cancer, RNAscope® in situ detection of HPV E6/E7 was performed and found to be positive in all instances where such material was available (n=12). Non-recurrent controls for the LRR and DMR cases were first screened for having follow-up at least as long as the latest LRR or DMR events. Controls were matched for the following features: 8th ed. AJCC pathologic T stage, N stage, and overall stage; smoking history >10 years; adjuvant radiation; chemotherapy drug. For LRRs, a second set of controls was selected from patients who did not recur despite refusing recommended adjuvant radiation, thus allowing comparison of LRR cases to an unusually treatment-sensitive control phenotype. The three case-control cohorts are illustrated in FIG. 6: (1) LRRs vs. controls completing definitive therapy (1:2 match); (2) LRR vs. controls refusing indicated adjuvant therapy (1:1 match); (3) DMRs vs. controls completing definitive therapy (1:1 match). In rare instances when a perfect match for T/N stage or smoking history were unavailable, the closest controls were used. Likewise, a control was used as a match for two cases if not enough unique controls were available.

A training data set was created based on clinical annotations and matching (anonymized) digital diagnostic pathology whole slide images of hematoxylin and eosin stained tumors. A virtual slide is a high-definition, fully digital capture of pathological specimen which can be used for pathological evaluation without significant loss of fidelity, scanned at a resolution of 0.25 μm per pixel. Tumor regions are digitally annotated on each virtual slide by a pathologist using digital pathology analysis platform QuPath. A tumor tile is a virtual tile sub-image focused only on areas of tumor, and with reduced focal depth to minimize file size and promote processing. The deep learning pipeline automatically and efficiently creates tumor tiles for each tumor region based on a specified tile size using software of our own development.

A deep convolutional neural network optimized to pathology imaging is then used to extract features from tumor tiles. During this computationally-intensive process, the neural network “learns” combinations of histology features most characteristic of a specified objective (in this case which cancers are most likely to recur). To accomplish this task, pixel data from extracted image tiles were normalized and then trained via a Tensorflow/Keras implementation of the Xception neural network model, with weights initialized using pretraining (e.g., using ImageNet).

In order to reduce bias against sparse categories, training batches were filled with tiles in a manner that was balanced according to the output category. At the time of training, tiles were randomly vertically and horizontally flipped, as well as randomly rotated 90, 180, or 270 degrees. Training performance was evaluated on a validation dataset chosen at the time of training using K-fold cross-validation, averaged across the folds. Using the algorithm on pilot data, an average cross-validated AUC of 0.91 was achieved, which is early evidence of strong predictive performance.

IV. Example Operations

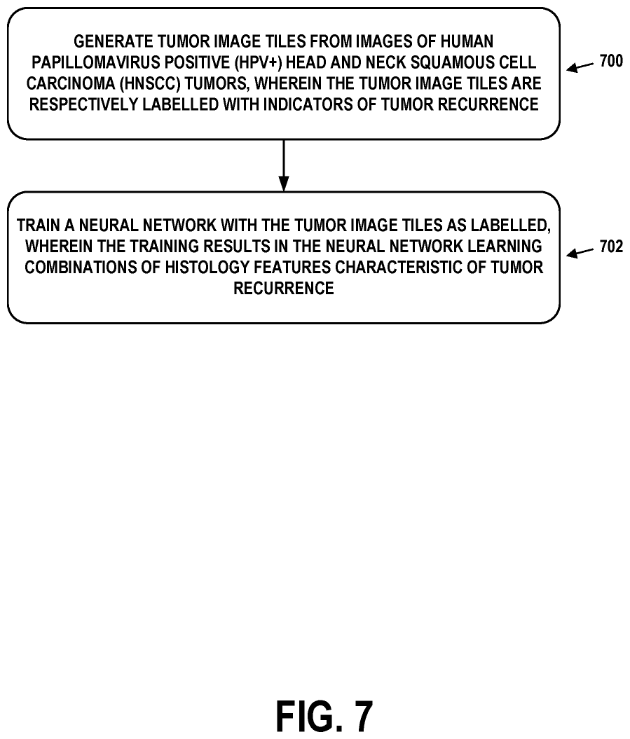

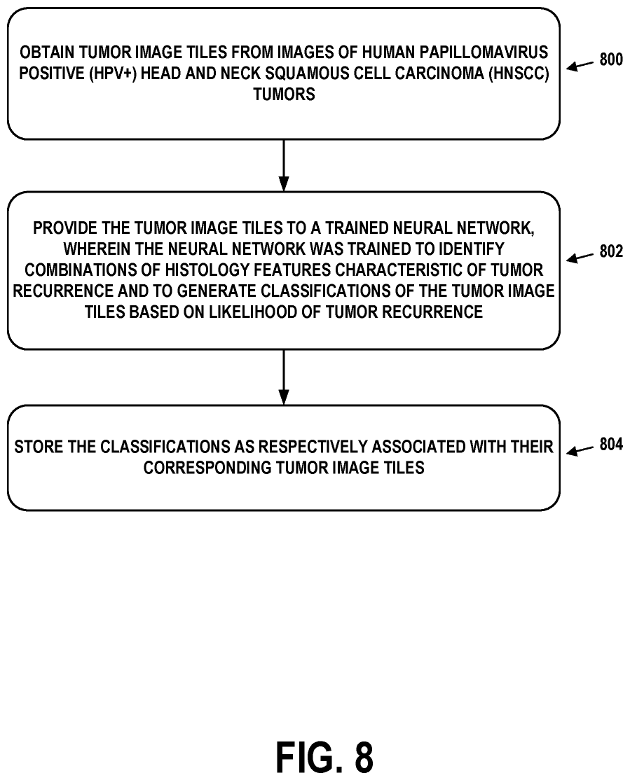

FIGS. 7 and 8 are flow charts illustrating example embodiments. The processes illustrated by FIGS. 7 and 8 may be carried out by a computing device, such as computing device 100, and/or a cluster of computing devices, such as server cluster 200. However, the processes can be carried out by other types of devices or device subsystems. For example, the processes could be carried out by a portable computer, such as a laptop or a tablet device.

The embodiments of FIGS. 7 and 8 may be simplified by the removal of any one or more of the features shown therein. Further, these embodiments may be combined with features, aspects, and/or implementations of any of the previous figures or otherwise described herein.

Block 700 of FIG. 7 may involve generating tumor image tiles from images of HPV+ HNSCC tumors, wherein the tumor image tiles are respectively labelled with indicators of tumor recurrence. These labels may indicate whether the tumor image tiles depict recurrent or non-recurrent tumors, for example.

Block 702 may involve training a neural network with the tumor image tiles as labelled, wherein the training results in the neural network learning combinations of histology features characteristic of tumor recurrence.

In some embodiments, the images are of hematoxylin and eosin stained tumors.

In some embodiments, training the neural network comprises normalizing pixel data from the tumor image tiles.

In some embodiments, training the neural network comprises applying a Tensorflow and Keras implementation of an Xception neural network model with weights initialized using pretraining.

In some embodiments, training the neural network comprises randomly vertically and horizontally flipping the tumor image tiles.

In some embodiments, training the neural network comprises randomly rotating the tumor image tiles by 90, 180, or 270 degrees.

In some embodiments, training the neural network comprises applying random JPEG compression or random Gaussian blur to the tumor image tiles. For instance, a random proportion of images may undergo JPEG compression at a random quality level between 50-100%. This JPEG compression augmentation may help improve generalizability as models are applied to a large variety of slide scanners and image formats. Alternatively or additionally, a random proportion of images may undergo a random amount of Gaussian blur. As above, this may help improve generalizability of models to slides that are slightly out of focus.

In some embodiments, training the neural network comprises determining batches of the tumor image tiles to use for training in a manner that is balanced according to the respective labels.

In some embodiments, the neural network is a deep convolutional neural network or a vision transformer network. Vision transformer networks involve dividing an image into patches and providing linear embeddings of these patches to a transformer-based network, where the patches are treated similarly to words when the transformer is used in a natural language processing context.

Block 800 of FIG. 8 may involve obtaining tumor image tiles from images of human papillomavirus positive (HPV+) head and neck squamous cell carcinoma (HNSCC) tumors.

Block 802 may involve providing the tumor image tiles to a trained neural network, wherein the neural network was trained to identify combinations of histology features characteristic of tumor recurrence and to generate classifications of the tumor image tiles based on likelihood of tumor recurrence.

Block 804 may involve storing the classifications as respectively associated with their corresponding tumor image tiles.

In some embodiments, the tumor image tiles are generated from images of hematoxylin and eosin stained tumors.

In some embodiments, the neural network was trained based on normalized pixel data from the tumor image tiles.

In some embodiments, the neural network was trained by applying a Tensorflow and Keras implementation of an Xception neural network model with weights initialized using pretraining.

In some embodiments, the neural network was trained by randomly vertically and horizontally flipping the tumor image tiles.

In some embodiments, the neural network was trained by randomly rotating the tumor image tiles by 90, 180, or 270 degrees.

In some embodiments, training the neural network comprises applying random JPEG compression or random Gaussian blur to the tumor image tiles. For instance, a random proportion of images may undergo JPEG compression at a random quality level between 50-100%. This JPEG compression augmentation may help improve generalizability as models are applied to a large variety of slide scanners and image formats. Alternatively or additionally, a random proportion of images may undergo a random amount of Gaussian blur. As above, this may help improve generalizability of models to slides that are slightly out of focus.

In some embodiments, the neural network was trained by determining batches of the tumor image tiles to use for training in a manner that is balanced according to respective labels.

In some embodiments, the neural network is a deep convolutional neural network or a vision transformer network.

V. Conclusion

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer readable medium such as a storage device including RAM, a disk drive, a solid state drive, or another storage medium.

The computer readable medium can also include non-transitory computer readable media such as non-transitory computer readable media that store data for short periods of time like register memory and processor cache. The computer readable media can further include non-transitory computer readable media that store program code and/or data for longer periods of time. Thus, the non-transitory computer readable media may include secondary or persistent long term storage, like ROM, optical or magnetic disks, solid state drives, compact-disc read only memory (CD-ROM), for example. The non-transitory computer readable media can also be any other volatile or non-volatile storage systems. A non-transitory computer readable medium can be considered a computer readable storage medium, for example, or a tangible storage device.

Moreover, a step or block that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

1. A computer-implemented method comprising:
generating tumor image tiles from images of human papillomavirus positive (HPV+) head and neck squamous cell carcinoma (HNSCC) tumors, wherein the tumor image tiles are respectively labelled with indicators of tumor recurrence; and
training a neural network with the tumor image tiles as labelled, wherein the training results in the neural network learning combinations of histology features characteristic of tumor recurrence, wherein training the neural network comprises applying random image compression or adding random Gaussian blur to the tumor image tiles.

2. The computer-implemented method of claim 1, wherein the tumor image tiles are of hematoxylin and eosin stained tumors.

3. The computer-implemented method of claim 1, wherein training the neural network comprises normalizing pixel data from the tumor image tiles.

4. The computer-implemented method of claim 1, wherein training the neural network comprises applying a Tensorflow and Keras implementation of an Xception neural network model with weights initialized using pretraining.

5. The computer-implemented method of claim 1, wherein training the neural network comprises randomly vertically and horizontally flipping the tumor image tiles.

6. The computer-implemented method of claim 1, wherein training the neural network comprises randomly rotating the tumor image tiles by 90, 180, or 270 degrees.

7. (canceled)

8. The computer-implemented method of claim 1, wherein training the neural network comprises determining batches of the tumor image tiles to use for training in a manner that is balanced according to the respective labels.

9. The computer-implemented method of claim 1, wherein the neural network is a deep convolutional neural network or a vision transformer network.

10. A computer-implemented method comprising:
obtaining tumor image tiles from images of human papillomavirus positive (HPV+) head and neck squamous cell carcinoma (HNSCC) tumors;
providing the tumor image tiles to a trained neural network, wherein the neural network was trained to identify combinations of histology features characteristic of tumor recurrence and to generate classifications of the tumor image tiles based on likelihood of tumor recurrence, wherein training the neural network involved applying random image compression or adding random Gaussian blur to the tumor image tiles; and
storing the classifications as respectively associated with their corresponding tumor image tiles.

11. The computer-implemented method of claim 10, wherein the tumor image tiles are generated from images of hematoxylin and eosin stained tumors.

12. The computer-implemented method of claim 10, wherein the neural network was trained based on normalized pixel data from the tumor image tiles.

13. The computer-implemented method of claim 10, wherein the neural network was trained by applying a Tensorflow and Keras implementation of an Xception neural network model with weights initialized using pretraining.

14. The computer-implemented method of claim 10, wherein the neural network was trained by randomly vertically and horizontally flipping the tumor image tiles.

15. The computer-implemented method of claim 10, wherein the neural network was trained by randomly rotating the tumor image tiles by 90, 180, or 270 degrees.

16. (canceled)

17. The computer-implemented method of claim 10, wherein the neural network was trained by determining batches of the tumor image tiles to use for training in a manner that is balanced according to respective labels.

18. The computer-implemented method of claim 10, wherein the neural network is a deep convolutional neural network or a vision transformer network.

19. A non-transitory computer-readable medium, having stored thereon program instructions that, upon execution by a computing device, cause the computing device to perform operations comprising:
obtaining tumor image tiles from images of human papillomavirus positive (HPV+) head and neck squamous cell carcinoma (HNSCC) tumors;
providing the tumor image tiles to a neural network, wherein the neural network was trained to identify combinations of histology features characteristic of tumor recurrence and to generate classifications of the tumor image tiles based on likelihood of tumor recurrence, wherein training the neural network involved applying random image compression or adding random Gaussian blur to the tumor image tiles; and
storing the classifications as respectively associated with their corresponding tumor image tile.

20. (canceled)

21. The computer-implemented method of claim 1, wherein the random image compression is random JPEG compression at a random quality level between 50% and 100%.

22. The computer-implemented method of claim 1, wherein adding random Gaussian blur to the tumor image tiles comprises applying the random Gaussian blur to a random proportion of the tumor image tiles.

23. The computer-implemented method of claim 1, wherein the tumor image tiles comprise virtual tile sub-images of tumor areas with reduced focal depth.
